(19) United States
(12) Patent Application Publication
(10) Pub. No.: US 2024/0408210 A1
(43) Pub. Date: Dec. 12, 2024

(54) Topical Compositions and Methods for Making and Using the Same

(71) Applicant: Noor Brands Company, LLC, High Point, NC (US)

(72) Inventors: Adnan Mjalli, High Point, NC (US); Wiley William Hitchcock, Grensboro, NC (US)

(21) Appl. No.: 18/738,969

(22) Filed: Jun. 10, 2024

Related U.S. Application Data
(60) Provisional application No. 63/471,886, filed on Jun. 8, 2023.

(51) Int. Cl. A61K 47/18; A61K 31/167; A61K 31/197; A61K 31/4375; A61K 31/522; A61K 47/10; A61P 29/00

(52) U.S. Cl. CPC A61K 47/186; A61K 31/167; A61K 31/197; A61K 31/4375; A61K 31/522; A61K 47/10; A61P 29/00

(57) ABSTRACT

Provided are topical compositions for the transdermal delivery of active agents and methods for making and using the same. The disclosed compositions are water-based formulations comprising a dual-surfactant blend that can be applied to the skin as a foam and used to for transdermal delivery active agents.

### RELATED APPLICATIONS

This application claims priority to U.S. provisional patent application No. 63/471,886, filed Jun. 8, 2023. The disclosure of U.S. provisional patent application No. 63/471,886 is incorporated by reference in its entirety herein.

### FIELD

The present disclosure relates to topical compositions for the transdermal delivery of active agents and methods for making and using the same.

### BACKGROUND

Transdermal delivery of active agents advantageously allows for targeted treatment of particular parts of the body and is beneficial over alternative routes of administration (e.g., oral, intravenous) due to fewer adverse side effects, ease of administration, and the ability to provide prolonged and controlled delivery of active agents. Additionally, transdermal delivery allows for active agent compounds to exert an effect beyond the local site of application. transdermal delivery avoids the gastrointestinal environment, which can affect the efficacy of active agents.

Compounds are commonly applied to the skin for a variety of purposes including treatment of local conditions and cosmetic enhancement. However, most topically applied compounds are poorly absorbed, and thus, cannot be transdermally delivered depending on the size and polarity of the compounds. Several factors determine whether an active agent can be delivered transdermally, including the properties of the active agents, the properties of the carrier of the active agents, and the properties of the topical site of application. The skin serves as a barrier to transdermal delivery of active agents based off the size and polarity of the active agents.

Thus, there is a need to develop transdermal delivery systems that can penetrate the skin to allow active agents to bypass the skin's lipid bilayer. The development of carrier formulations capable of diffusing the skin to create pores or channels in the skin's lipid bilayer's through which the active ingredients can pass is advantageous.

### SUMMARY OF THE DISCLOSURE

The use of a carrier composition comprising a dual surfactant blend presents a promising strategy for transdermal delivery of active agents. In a first aspect, disclosed herein is a composition formulated for topical application comprising: (i) a carrier comprising a surfactant and (ii) an active agent blend. In some embodiments, the surfactant is a dual surfactant blend. In some embodiments, the dual surfactant blend comprises benzalkonium chloride and dihydroxyethyl cocamine oxide. In certain embodiments, the active agent blend comprises one at least one active ingredient useful for improving health and fitness. In certain embodiments, the active agent blend comprises one at least one active ingredient useful for preventing and/or relieving pain. In certain embodiments, the active agent blend comprises one at least one active ingredient useful for joint and health repair. In certain embodiments, the active agent blend comprises one at least one active ingredient useful for preventing and/or treating wrinkles. In yet other embodiments, the active agent blend comprises one at least one active ingredient useful for subcutaneous fat reduction. In some embodiments, the active agent blend comprises one at least one active ingredient useful for breast enhancement. In some embodiments, the active agent blend comprises one at least one active ingredient useful for or preventing and/or treating erectile dysfunction. In some embodiments, the active agent blend comprises one or more of L-Arginine, caffeine, yohimbine bark extract, yohimbine HCl, collagen peptide, hyaluronic acid, aloe vera powder, Cissus quadrangularis, forskolin, fenugreek and lidocaine hydrochloride. Or other active agents as disclosed herein may be used.

In a second aspect, disclosed is a method of using any of the disclosed compositions comprising topically administering the composition to the skin of a subject thereby delivering at least one active agent across the skin. In an embodiment, the composition comprises (i) a carrier comprising a surfactant and (ii) an active agent blend. In some embodiments, the surfactant is a dual surfactant blend. In some embodiments, the dual surfactant blend comprises benzalkonium chloride and dihydroxyethyl cocamine oxide.

In a third aspect, discloses is a method of preparing any of the disclosed compositions comprising: (i) preparing a carrier comprising a surfactant, (ii) preparing an active agent blend: and (iii) combining the carrier and the active agent blend. In some embodiments, the surfactant is a dual surfactant blend. In some embodiments, the dual surfactant blend comprises benzalkonium chloride and dihydroxyethyl cocamine oxide.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 depicts a schematic representation of a method for preparing a topical composition in accordance with an embodiment of the disclosure.

### DETAILED DESCRIPTION

The presently disclosed subject matter now will be described more fully hereinafter with reference to the accompanying description, in which some, but not all embodiments of the presently disclosed subject matter are shown. The disclosed subject matter can be embodied in many different forms and should not be construed as limited to the embodiments set forth herein: rather, these embodiments are provided so that this disclosure will satisfy applicable legal requirements. Like numbers refer to like elements throughout.

Many modifications and other embodiments of the disclosed subject matter set forth herein will come to mind to one skilled in the art to which the disclosed subject matter pertains having the benefit of the teachings presented in the description. Therefore, it is to be understood that the disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation.

### Terms and Definitions

In order for the disclosure to be more readily understood, certain terms are first defined. Additional definitions for the following terms and other terms are set forth throughout the specification. While the following terms are believed to be well understood by one of ordinary skill in the art, the following definitions are set forth to facilitate explanation of the presently disclosed subject matter. Unless otherwise defined, all technical and scientific terms used herein have the same meaning as commonly understood by one of ordinary skill in the art to which this presently described subject matter belongs. Additionally, any reference referred to as being “incorporated herein” is to be understood as being incorporated in its entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

It is further noted that, as used in this specification, the singular forms “a.” “an,” and “the” include plural referents unless expressly and unequivocally limited to one referent. The term “and/or” generally is used to refer to at least one or the other. In some cases, the term “and/or” is used interchangeably with the term “or.” The term “including” is used herein to mean, and is used interchangeably with, the phrase “including but not limited to.” The term “such as” is used herein to mean, and is used interchangeably with, the phrase “such as but not limited to.”

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Thus, the term “about” Is used to indicate that a value includes the inherent variation of error for the device, the method being employed to determine the value, or the variation that exists among samples.

The term “buffer solution” is used to indicate a solution that resists a change in pH hen hydrogen ions (H⁺) or hydroxide ions (OH⁻) are added. A buffered solution may be produced by mixing a weak acid with its conjugate base. The buffer solution may be added to water to create “buffered water.”

The term “therapeutically effective amount” refers to an amount effective, at dosages and for periods of time necessary, to achieve a desired therapeutic result. A therapeutically effective amount may be the amount of a compound that results in at least one of prevention or delay of onset or amelioration of symptoms associated with a disease, disorder, or condition in a subject or an attainment of a desired biological effect (e.g., cosmetic or performance enhancement or improvement).

The term “topical” or topically” is used to refer to administration or application of the composition to a defined area of the body such as a defined area of skin surface.

The term “transdermal” or “transdermally” refers to the penetration and movement of a biologically active agent through the epidermis and dermis, or epidermis, dermis and hypodermis. Transdermal administration can be accomplished by applying. pasting, rolling, attaching, pouring, pressing, rubbing, etc., of a transdermal preparation onto a skin surface.

The term “solvent” is used to refer to any substance, typically a liquid, in which other substances dissolve.

The term “subject” and “individual” may be used interchangeably. A subject may comprise an animal. Thus, in some embodiments, the subject is a mammalian animal, including, but not limited to a dog, a cat, a horse, a rat, a monkey, and the like. In some embodiments, the subject is a human subject. In some embodiments, the subject is a patient, that is, a living person presenting themselves in a clinical setting for diagnosis, prognosis, or treatment of a disease or condition.

### Compositions

In a first aspect, disclosed herein is a composition formulated for topical application comprising: (i) a carrier comprising a dual surfactant blend and (ii) an active agent blend. In some embodiments, the dual surfactant blend comprises benzalkonium chloride and dihydroxyethyl cocamine oxide. In some embodiments, the composition is a water-based solution comprising a dual-surfactant blend that is delivered as a foam and used to apply a topical active agent for transdermal delivery.

### Carriers

In one aspect, provided herein is a composition comprising a dual surfactant carrier, wherein the carrier comprises a benzalkonium chloride (BZK) and Dihydroxyethyl Cocamine Oxide (MACAT). As used herein “BZK” is used interchangeably with alternate abbreviations for benzalkonium chloride, which may include BKC, BAK, or BAC. In certain embodiments, the carrier is a dual-surfactant blend. The carrier composition may, in certain embodiments, further comprise one or more of water or buffered water, glycerin, and PEG-400. In some embodiments, the carrier composition is free of alcohol. Such a composition is advantageous in that it is safe for topical use, stable, and possesses antimicrobial activity.

BZK's are a class of quaternary ammonium compounds that include a benzene ring and a nitrogen constituent (i.e., a quaternary ammonium group) near the ring. A carbon atom is disposed between the nitrogen constituent and the benzene ring. Two methyl groups and an R group of varying size extend from the nitrogen atom. A suitable BZK (e.g., CAS #68391 Jan. 5, “Nexsurf BZK”) may be obtained from many commercial suppliers. BZK's are traditionally used as preservatives, antiseptics, and/or disinfectants. BZK's are commonly found in hand sanitizers and cleaning products.

MACAT is a tertiary amine oxide with functions including as a conditioning agent, surfactant, antistatic agent, and foaming agent. A MACAT suitable for use in the disclosed composition (CAS #61791-47-7) may be obtained from many commercial suppliers such as Mason Chemical Co.

In certain embodiments, the carrier may minimize any degradation of the active agents and/or may minimize any adverse effects from the active ingredients in a subject. In certain embodiments, the carrier may comprise a diluent, a solubilizer, an emulsifier, a preservative, and/or an adjuvant. In some embodiments, the carrier further comprises one or more of water or buffered water, glycerin, and PEG-400. Thus, in certain embodiments, the carrier comprises BZK, MACAT, water, glycerin, and PEG-400.

In some embodiments, the BZK is less than or equal to 0.5, 0.4, 0.3, 0.2, or 0.1% by weight of the total weight of the composition. In some embodiments, the BZK is about 0.2, 0.21, 0.22, 0.23, 0.24, 0.25, 0.26, 0.27, 0.28, 0.29, or 0.3% by weight of the total weight of the composition. Thus, in certain embodiments, the BZK is from 0.02-2%, or 0.05%-1%, or 0.1-0.5%, or 0.2-0.3% or subranges therein by weight.

In some embodiments, the MACAT is less than or equal to 0.5, 0.4, 0.3, 0.2,or 0.1% by weight of the total weight of the composition. In some embodiments, the MACAT is about 0.2. 0.21, 0.22, 0.23, 0.24, 0.25, 0.26, 0.27, 0.28, 0.29, or 0.3% by weight of the total weight of the composition. Thus, in certain embodiments, the MACAT is from 0.02-2%, or 0.05%-1%, or 0.1-0.5%, or 0.2-0.3% or subranges therein by weight.

In some embodiments the glycerin is less than or equal to 5.0, 4.5, 4.0, 3.5,3.0, 2.5, 2.0, 1.5, 1.0, or 0.5% by weight of the total weight of the composition. In some embodiments, the glycerin is about 1.0, 1.1, 1.2, 1.3, 1.4, 1.5, 1.6, 1.7, 1.8, 1.9, or 2.0% by weight of the total weight of the composition. Thus, in certain embodiments, the glycerin is from 0.2-10%, or 0.5-5%, or 1.0%-4.0%, or 1.0-3.0%, or 1.0-2.0% or subranges therein by weight.

In some embodiments the PEG-400 is less than 5.0, 4.5, 4.0, 3.5, 3.0, 2.5, 2.0, 1.5, 1.0, or 0.5% by weight of the total weight of the composition. In some embodiments, the PEG-400 is about 1.5, 1.6, 1.7, 1.8, 1.9, 2.0, 2.1, 2.2, 2.3, 2.4, or 2.5% by weight of the total weight of the composition. Thus, in certain embodiments, the PEG-400 is from 0.2-10%, or 0.5-5%, or 1.0-4.0%, or 1.0-3.0%, or 1.0-2.5% or subranges therein by weight.

In some embodiments, the water (e.g., diH₂O, deionized H₂O) is less than or equal to 70, 75, 80, 85, 90, 95%, or 98% weight of the total weight of the composition. In some embodiments, the water is about 88, 88.5, 89, 89.5, 90, 90.5, 91, 91.5 or 92.0% weight of the total weight of the composition. In some embodiments the water is about 89.70, 89.71, 89.72, 89.73, 89.74, 89.75, 89.76, 89.77, 89.78, 89.79, or 89.80% weight of the total weight of the composition. Thus, in certain embodiments, the water is from 60-97%, or 70-95%, or 85-90%, or 88-92%, or 89-90% or subranges therein by weight.

In certain instances, the carrier composition is a foam capable of delivery one or more active agents. These active agents may be useful for a variety of purposes, including but not limited to improving health and fitness, preventing and/or relieving pain, and/or joint and health repair, and/or preventing and/or treating wrinkles, and/or subcutaneous fat reduction, and/or breast enhancement, or preventing and/or treating erectile dysfunction.

### Active Agent Blends

In some embodiments, the composition further comprises an active agent blend. In certain embodiments, the active agent blend comprises 1, 2, 3, 4, 5, 6, 7, 8, 9, 10, 11, 12, 13, 14, 15 or more active agents. Active agents are compounds having therapeutic or cosmetic activity. For example, in some embodiments, the active agent has analgesic, anti-inflammatory, athletic performance-enhancing, anti-aging, wrinkle-reducing, breast-enhancing, and/or lipolytic effects.

In some embodiments the active agent blend comprises one or more of L-Arginine, caffeine, Yohimbine HCl, Yohimbine bark extract, collagen peptides, hyaluronic acid, aloe vera powder, Cissus quadrangularis, Pueraria mirifica, fenugreek, forskolin, COAXEL™ pH, or lidocaine HCl.

L-Arginine HCl is the hydrochloride salt form of L-arginine, an amino acid naturally found in protein-rich foods, including fish, red meat, poultry, soy, beans, and dairy. L-Arginine is a naturally occurring functional amino acid present in foods. L-arginine is a precursor to nitric oxide, which promotes vasodilation and increases blood flow: L-arginine also stimulates the release of growth hormone, insulin, and other substances in the body. L-Arginine acts as a vasodilator and stimulates the immune system, in part, by increasing production of nitric oxide. L-arginine is commonly used to treat a variety of conditions, including angina, hypertension, preeclampsia, erectile dysfunction, and peripheral arterial disease. In some embodiments, the L-Arginine is less than or equal to 10, 9, 9, 8, 7, 6, 5, 4, 3, 2, or 1% by weight of the total weight of the composition. In some embodiments, the L-Arginine is about 4.0, 4.5, 5.0, 5.5, 6.0, 6.5, 7.0, 7.5, or 8.0% by weight of the total weight of the composition. Thus, in certain embodiments, the L-Arginine HCl is from 0.5-20%, or 1-10%, or 2-8%, or 4-8%, or 5-6% or subranges therein by weight.

Caffeine is a purine, a methylxanthine alkaloid and acts primarily as an adenosine receptor antagonist with anti-inflammatory activity and neurostimulation. Caffeine promotes lipolysis by inhibiting phosphodiesterase. Caffeine also has stimulant effects and is known to increase metabolism. In some embodiments, the caffeine is less than or equal to 5.0, 4.0, 3.0, 2.0, 1.0, 0.75, 0.5, 0.25, 0.1% by weight of the total weight of the composition. In some embodiments, the caffeine is about 3.0, 2.5, 2.0, 1.5, 1.0, 0.75, 0.5, or 0.25% by weight of the total weight of the composition. Thus, in certain embodiments, the caffeine is from 0.05-10%, or 0.1-5.0%, or 0.25-3.0%, or 1.0-2.0%, or 0.5-1.5% or subranges therein by weight.

Yohimbine (YHM) is an indole alkaloid derived from the bark of the Pausinystalia johimbe tree. Yohimbine HCl is an alpha-2-adrenoceptor antagonist (i.e., a2 blocker) that causes vasodilation when applied locally. Mechanistically, α2 blockers may increase adrenergic, dopaminergic and serotonergic neurotransmitters and can induce insulin secretion, decreasing blood sugar levels. YHM HCl is a commercially available, standardized form of YHM commonly used as a dietary supplement. Additionally, YHM and YHM HCl may be useful for treating erectile dysfunction, improving athletic performance, and weight loss. In some embodiments, the YHM HCl is less than 5.0, 1.0, 0.9, 0.8, 0.7, 0.6, 0.5, 0.4, 0.3, 0.2, or 0.1% by weight of the total weight of the composition. In some embodiments, the YHM HCl is about 0.5, 0.45, 0.40, 0.35, 0.30, 0.25, 0.20, 0.15, 0.1, or 0.05% by weight of the total weight of the composition. Thus, in certain embodiments, the YHM HCl is from 0.05-5%, 0.05-1.0%, or 0.05-0.5%, or 0.1-0.4%, or 0.2-0.3% or subranges therein by weight. In some embodiments, the YHM is less than 0.1, 0.09, 0.08, 0.07, 0.06, 0.05. 0.04, 0.03, 0.03, 0.01, 0.005% by weight of the total weight of the composition. In some embodiments, the YHM is about 0.05, 0.04, 0.03, 0.02, or 0.01% by weight of the total weight of the composition. Thus, in certain embodiments, the YHM is from 0.005-0.1%, or 0.01-0.08%, 0.01-0.03%, or 0.015-0.025% or subranges therein by weight.

Collagen peptides are pieces of protein from animal collagen and act to stimulate and volumize skin and provide peptides for rebuilding skin collagen. In certain embodiments, the composition comprises commercially available collagen peptides, hydrolyzed Type I and III collagen, food grade (GrovyBee.com). In some embodiments, the collagen peptide is less than 2.0, 1.5 1.0, 0.75, 0.5, 0.25, 0.1% by weight of the total weight of the composition. In some embodiments, the collagen peptide is about 1.0, 0.9, 0.8, 0.7, 0.6, 0.5, 0.4, 0.3, 0.2, or 0.1% by weight of the total weight of the composition. Thus, in certain embodiments, the collagen peptide is from 0.05-5%, or 0.1-2.0%, or 0.1-1.0%, or 0.2-0.8%, or 0.4-0.6% or subranges therein by weight.

Hyaluronic acid (HA) is a naturally occurring glycosaminoglycan found throughout the body's connective tissue. HA can provide structure to skin, has anti-aging and anti-wrinkle effects, and can increase skin elasticity. HA has also been shown to have wound healing properties. HA is commonly used in skin care products and cosmetics. In some embodiments, the HA is less than 2.0, 1.5 1.0, 0.75, 0.5, 0.25, 0.1, 0.05, 0.01, or 0.005% by weight of the total weight of the composition. In some embodiments, the HA is about 0.5, 0.45, 0.40, 0.35, 0.30, 0.25, 0.20, 0.15, 0.10, 0.05, 0.01, or 0.005% by weight of the total weight of the composition. Thus, in certain embodiments, the HA is from 0.002-5%, or 0.005-2.0%, or 0.005-1.0%, or 0.005-0.5%, or 0.1-0.4% or 0.05-0.25% or subranges therein by weight.

Aloe vera is a succulent plant species. The leaves of the aloe vera plant can be dehydrated and ground into aloe vera powder, which is commonly used in cosmetic and therapeutic products for its moisturizing and cooling properties. In some embodiments, the aloe vera powder is less than 2.0, 1.5 1.0, 0.75, 0.5, 0.25, 0.1, 0.05, 0.01, or 0.005% by weight of the total weight of the composition. In some embodiments, the aloe vera powder is about 0.5, 0.45, 0.40, 0.35, 0.30, 0.25, 0.20, 0.15, 0.10, 0.05, 0.01, or 0.005% by weight of the total weight of the composition. Thus, in certain embodiments, the aloe vera is from 0.002-5%, or 0.005-2.0%, or 0.005-1.0%, or 0.005-0.5%, or 0.1-0.4% or 0.05-0.25% or subranges therein by weight.

Cissus quadrangularis (Cissus Q) is a vine with antioxidant, analgesic, and anti-inflammatory properties. Cissus Q has may be useful for weight loss and treating joint pain. In some embodiments, the Cissus Q is less than 2.0, 1.5 1.0, 0.75, 0.5, 0.3, 0.1, 0.05, 0.01, or 0.005% by weight of the total weight of the composition. In some embodiments, the Cissus Q is about 0.5, 0.45, 0.40, 0.35, 0.30, 0.25, 0.20, 0.15, 0.10, 0.05, 0.01, or 0.005% by weight of the total weight of the composition. Thus, in certain embodiments, the Cissus Q is from 0.002-5%, 0.005-2.0%, or 0.005-1.0%, or 0.005-0.5%, or 0.1-0.4% or 0.05-0.25% or subranges therein by weight.

Pueraria mirifica (PM) is a plant that is believed to contain isoflavones—a pant chemical with estrogen-like activity. PM has been used in traditional medicine for its anti-aging, anti-wrinkle, and breast-enhancing effects. PM has been used for weight loss and joint pain. In some embodiments, the PM is less than 2.0, 1.5 1.0, 0.8, 0.6, 0.4, 0.2, 0.1, 0.05, 0.01, or 0.005% by weight of the total weight of the composition. In some embodiments, the PM is about 0.8, 0.75, 0.60, 0.65, 0.60, 0.55, 0.50, 0.45, 0.40, 0.35, 0.3, or 0.25% by weight of the total weight of the composition. Thus, in certain embodiments, the PM is from 0.002-5%, or 0.005-2.0%, or 0.005-1.0%, or 0.005-0.5%, or 0.1-0.4% or 0.05-0.25% or subranges therein by weight.

Fenugreek is a clover-like herb. Fenugreek seeds are a dietary supplement believed to have lipolytic effects. In some embodiments, the fenugreek is less than 2.0, 1.5 1.0, 0.75, 0.5, 0.3, 0.1, 0.05, 0.01, or 0.005% by weight of the total weight of the composition. In some embodiments, the fenugreek is about 0.5, 0.45, 0.40, 0.35, 0.30, 0.25, 0.20, 0.15, 0.10, 0.05, 0.01, or 0.005% by weight of the total weight of the composition. Thus, in certain embodiments, the fenugreek is from 0.002-5%, or 0.005-2.0%, or 0.005-1.0%, or 0.005-0.5%, or 0.1-0.4% or 0.05-0.25% or subranges therein by weight.

Forskolin is made from the root plant in the mint family and has historically been used in traditional medicine for a variety of purposes, including weight loss. In some embodiments, the forskolin is less than 2.0, 1.5 1.0, 0.75, 0.5, 0.25, 0.1, 0.05, 0.01, or 0.005% by weight of the total weight of the composition. In some embodiments, the forskolin is about 0.8, 0.75, 0.60, 0.65, 0.60, 0.55, 0.50, 0.45, 0.40, 0.35, 0.3, or 0.25% by weight of the total weight of the composition. Thus, in certain embodiments, the forskolin is from 0.002-5%, or 0.005-2.0%, or 0.005-1.0%, or 0.005-0.5%, or 0.1-0.4% or 0.05-0.25% or subranges therein by weight.

COAXEL™ pH contains caffeine and is a slimming and conditioning agent useful in promoting lipolysis. In some embodiments, the COAXEL™ pH is less than 10, 9, 8, 7, 6, 5, 4, 3, 2, or 1% by weight of the total weight of the composition. In some embodiments, the COAXEL™ pH is about 7.0, 6.5, 6.0, 5.5, 5.0, 4.5, 4.0, 3.5 or 3.0% by weight of the total weight of the composition. Thus, in certain embodiments, the COAXEL™ pH is from 0.5-20%, or 1-10%, or 2-8%, or 3-7%, or 3-6% or 4-5% or subranges therein by weight.

Lidocaine HCl is the hydrochloride salt form of lidocaine, an aminoethylamide. Lidocaine HCl is a local anesthetic and cardiac depressant commonly used to numb an area of the body to reduce pain or discomfort. In some embodiments, the lidocaine HCl is less than 25, 20, 15, 14, 13, 12, 11, 10, 9, 9, 8, 7, 6, 5, 4, 3, 2, or 1% by weight of the total weight of the composition. In some embodiments, the lidocaine HCl is about 12.5, 12.0, 11.5, 11.0, 10.5, 10.0, 9.5, 9.0, 8.5, 8.0, 7.5 or 7.0% by weight of the total weight of the composition. Thus, in certain embodiments, the lidocaine HCl is from 0.5-30%, or 1-25%, or 1-20%, or 5-15%, or 7-12.5% or 8-11% or subranges therein by weight.

In certain embodiments, the active agent blend comprises 1, 2, 3, 4, 5, 6, 7, 8, 9, 10, 11, 12, 13, 14, 15 or more active agents. Any active agents suitable for transdermal administration may be delivered using the compositions described herein. Active agents are compounds having therapeutic or cosmetic activity. In some embodiments, the two or more active agents work synergistically. In some embodiments, the active agents are useful in preventing or treating one or more medical condition or illness. In some embodiments, the active agents are useful in cosmetic enhancement. For example, in certain embodiments, the topical composition can be used to topically deliver active agents for preventing or treating, wrinkles, pain, erectile dysfunction, breast enhancement, subcutaneous fat reduction. In some embodiments, the transdermal delivery system can be used to topically deliver active ingredients that promote health and fitness.

Generally, the amount of active agent blend in the topical composition can vary from about 1-20% by total weight of the composition. In some embodiments, the amount of active agent blend in the topical composition ranges from about 1-15% by weight, to 3-12%, to 4-10%, to 5-9%, or 6-8%.

In some embodiments, the composition is a topical formulation. In some embodiments, the composition is a topical formulation capable of delivering one or more active agents transdermally. In certain embodiments, the topical formulation is a foam.

Delivery of two or more active agents together and topically can be challenging due to the size and polarity of the agents. The unique properties of the foam formulations surprisingly allow for delivery of multiple active agents simultaneously. In certain embodiments, the two or more active agents comprise at least one water soluble agent. In some embodiments, the two or more active agents comprise at least one lipid soluble agent. In certain embodiments, the two or more active agents comprise at least one water soluble agent and at least one lipid soluble agent. Thus, the disclosed carrier composition/delivery system is advantageous in that it is capable of delivering at least 1, 2, 3, 4, 5, 6, 7, 8, 9, or 10 or more active agents simultaneously. Additionally, the carrier composition/delivery system is capable of delivering agents with differing solubility (e.g., lipid vs water) simultaneously.

In some embodiments the active agents are in powdered form. Thus, in some embodiments, the active agents are dissolved to form an active agent blend solution. In some embodiments, the powdered active agents may be dissolved using a primary non-aqueous solvent. In addition to being capable of dissolving the powdered active agents, the solvent may have other characteristics. Thus, in some embodiments, the solvent is suitable for biomedical uses, including skin contact, has low toxicity, and serves as a quick-drying agent. In some embodiments, the solvent is a lower alcohol. Lower alcohols are colorless liquids at normal temperatures and include ethanol, propanol, and isopropanol. In some embodiments, the solvent is ethanol. In other embodiments, the solvent is a liniment base. Any liniment base known in the art is suitable for use as a solvent. Examples of such liniment bases include mixtures composed of 10-70 parts by weight of an alcohol such as but not limited to a monohydric alcohol (e.g., ethanol, propanol, isopropyl alcohol), a polyhydric alcohol (e.g., polyethylene glycol, propylene glycol, butylene glycol, or the like), up to 55 parts by weight of water, up to 60 parts by weight of a fatty acid ester (e.g., an ester of adipic acid, sebacic acid, or myristic acid) and up to 10 parts by weight of a surfactant (e.g., polyoxyethylene alkyl ether). In certain embodiments, neutralizing agents (e.g., for pH adjustment), tackifiers (e.g., methyl cellulose, carboxyvinyl polymer, or hydroxypropyl cellulose), rash-preventing agents, and other additives (e.g., salicylic acid, methyl salicylate, glycol salicylate, 1-menthol, camphor: peppermint oil, capsicum, extract, nonylic vanillylamide, crotamiton, Azone, propylene carbonate, or diisopropyl adipate) may also be added in the liniment.

In some embodiments, the powdered active agents may be dissolved using dimethyl sulfoxide (DMSO), an organic form of sulfur. DMSO is currently used for the treatment of inflammatory conditions and cancer and has been demonstrated to be effective a topical agent. DMSO is able to both penetrate skin and aid dermal penetration of other therapeutics. Brien et al., Osteoarthritis and Cartilage (2008) 16:1277-1288. Thus, in some embodiments, DMSO functions as an analgesic, a solvent, and a facilitator of therapeutic transdermal delivery. In some embodiments, the topical composition comprises about 1-10%, about 2-5%, or about 2.5% DMSO by weight.

The stability of the formulation is an important factor in determining the suitability of the formulation for commercial use. In certain embodiments, the compositions described herein provide advantages including stability at 3 months, 6months, 1 year or more at 4-40° C. as reflected in the lack of changes in viscosity, color, precipitation, crystallization, and phase/layer separation, In certain embodiments, the disclosed foam formulations are able to maintain the active and functional agents without significant degradation over a period of time and over a range of temperatures. In some embodiments the composition is capable of being stored for at least 3 months, 6months, 1 year, 2 years, or 5 years or more at 4-40° C. without significant degradation. In some embodiments, the rate of degradation is less than 2.0, 1.0, 0.9, 0.8, 0.7, 0.6, 0.5, 0.4, 0.3, 0.2, or less than 0.1%, over the course of 6 or more months at room temperature. In some embodiments, the compositions disclosed herein remain stable following one or more freeze-thaw cycles.

### Methods of Use

In another aspect, provided herein is a method of using any of the compositions described herein comprising topically administering the composition to the skin of a subject, thereby delivering at least one active agent across the skin. In an embodiment, the composition comprises (i) a carrier comprising a dual surfactant blend and (ii) an active agent blend. In some embodiments, the dual surfactant blend comprises benzalkonium chloride and dihydroxyethyl cocamine oxide. In certain embodiments, the method comprises topically administering to the subject a composition comprising a therapeutically effective amount of one or more active agents. In certain embodiments, the methods may be used to preventing and/or treating a subject.

In some embodiments, the subject may be any animal. In certain embodiments, the subject is a human. In some embodiments, the subject has a disease, disorder, condition, or ailment. In some instances, for example, in some embodiments, the subject may be experiencing inflammation, pain, obesity, neurodegenerative disease, arthritis, skin condition (e.g., wrinkles), or erectile dysfunction. In certain embodiments, the subject may seek to enhance or improve breast size or appearance. In other embodiments, the subject may seek to improve athletic performance.

In some embodiments, the method of using a composition comprises topically administering to the subject any one of the compositions described herein to the skin or mucosal surface. Transdermal delivery can improve the efficiency and therapeutic bioavailability, in part, by avoiding the first-pass metabolism and gastrointestinal environment that impacts oral administration of active agents. Transdermal delivery of active agents primarily occurs via stratum corneum, which consists of dead, keratinized epidermal cells. Delivery of active agents across the epidermal cells can be challenging. Thus, in certain embodiments, the size and properties of the therapeutics as well as the properties of the delivery system determine whether a therapeutic can be delivered transdermally.

In some embodiments, the compositions described can be topically administered by rubbing the composition over an area of skin. In certain embodiments, the composition is rubbed over an area of the skin until the composition is adsorbed by and/or no longer detected on the surface of the skin. In some instances, the area of skin is in need of treatment thereof. For example, the composition may be applied to an area of skin having wrinkles. In certain other embodiments, the area of skin covers a region of the body in need of treatment thereof. For example, the composition may be applied to the skin covering an arthritic joint. Topical sites treatable through the use of the compositions described herein include, but are not limited to forehead, arms, legs, chest, back, neck, hands, feet.

The compositions can be applied by various physical means, including but not limited to applicator pads, swabs, roller bottles, or other devices capable of applying the compositions in a thin film. The compositions can be applied with any means known in the art so long as when applied to the area of the skin, the composition will stay in place, i.e., without run-off, for sufficient time, to permit an individual to spread and retain the composition over and on the affected area. In some embodiments, the topical composition is administered using a patch or bandage. In some embodiments, the composition is applied directly to the skin. In some instances, the applied composition is massaged into the skin. For example, following application, the composition may be massaged into the skin using 2-3 fingertips until the treated area is moist but not wet. In some embodiments, the applied composition is allowed to air-dry.

In some embodiments, the composition is applied preemptively, retroactively or both preemptively and retroactively. Thus, in some embodiments, the composition is used proactively to prevent pain and/or inflammation. In certain embodiments, the composition is used retroactively to treat pain and/or inflammation.

In some embodiments, the disclosed compositions provide certain advantages including drying time, ability to adhere to skin, spreadability, and greater absorption of active agents.

In some embodiments, the composition is applied preemptively, retroactively or both preemptively and retroactively. The composition may, in certain embodiments, be administered at least 1, 2, 3, 4, or 5 times a day for at least 1, 2, 3, 4, 5, 6, 7, 8, 9, 10, 11, or 12 days, or for several weeks, or months, or years. Or, longer periods of application may be used.

In some embodiments, the compositions are useful in improving health and fitness and/or athletic performance, pain relief, promoting joint health and repair, wrinkle treatment, breast enhancement, subcutaneous fat reduction, or erectile dysfunction.

### Health and Fitness

In some embodiments, the composition is used to improve health and fitness. In certain instances the method is used to activate muscles, stimulate joints, increase blood flow to the treated area, and/or increase sympathetic nervous activity. In some embodiments, the method is useful for decreasing next-day soreness and stiffness that may result from physical activity.

In certain instances, the method comprises applying an effective amount of a composition comprising an active agent blend topically to an area of the skin of a subject, wherein the active agent blend comprises at least one active ingredient useful for improving health and fitness. Active ingredients useful for improving health and fitness, include but are not limited to yohimbine HCl, caffeine, and arginine HCl.

In some embodiments, the composition is applied to an area of the skin prior to an athletic activity (e.g., exercise, athletic competition). In certain embodiments, the composition is applied less than 120, 90, 60, 30, 15, 10, or 5 minutes prior to the athletic activity. In some embodiments, the composition is applied after an athletic activity. In some embodiments, the composition is applied at least 5, 10, 15, 30, 60, 90, or 120 or more minutes following an athletic activity.

### Pain Relief

In some embodiments, the composition is used to relieve pain. In certain embodiments, the methods are used to alleviate acute pain, chronic pain, or both. In certain instances, the methods and compositions described herein may be used for treatment of pain, including but not limited to neck pain, back pain, joint pain, migraine pain, and muscular pain.

In some embodiments, the methods are useful for treating acute pain due to minor strains, sprains and contusions. In an embodiment of the application the pain is associated with inflammation. In a further embodiment the pain is associated with osteoarthritis. In certain embodiments, the compositions of the application are useful for the treatment of other pain-associated disorders, including but not limited to muscle pain, lower back pain, neck pain, rheumatoid arthritis, tendonitis, fibromyalgia, myofascial pain, carpal tunnel syndrome, gout and neuropathic pain conditions.

In certain instances, the method comprises applying an effective amount of a composition comprising an active agent blend topically to an area of the skin of a subject, wherein the active agent blend comprises at least one active ingredient useful for relieving pain. Active ingredients useful for relieving pain, include but are not limited to yohimbine HCl, caffeine, arginine HCl, Cissus Q. In some embodiments, the active ingredient useful for relieving pain has anesthetic properties. For example, the active agent blend may further comprise lidocaine.

In certain embodiments, the composition is applied as needed to relieve pain. In some embodiments, the composition is applied less than 120, 90, 60, 30, 15, 10, 5, or 1 min following a pain event.

### Joint Health & Repair

In some embodiments, the composition is used to improve joint health and/or repair joints. In certain embodiments, the compositions are useful for the treatment of chronic joint diseases characterized by joint pain, degeneration of articular cartilage, impaired movement, and stiffness. Suitable joints include, for example, the knee, elbow, hand, wrist, and hip.

In some instances, the method comprises applying an effective amount of a composition comprising an active agent blend topically to an area of the skin of a subject, wherein the active agent blend comprises at least one active ingredient useful for joint health and/or repair. Active ingredients useful for joint health repair, include but are not limited to yohimbine HCl, caffeine, L-arginine HCl, collagen peptides, Cissus quadrangularis, chondroitin sulfate sodium, glucosamine hydrochloride, methylsulphonylmethane (MSM), turmeric (curcumin), Boswellia serrata extract, and S-adenosyl-L-methionine (SAMe).

### Wrinkle Treatment

In some embodiments, the composition is used to prevent and/or treat wrinkles. In certain embodiments, the compositions are useful for minimizing wrinkles and/or other skin imperceptions. In certain embodiments, the methods are useful for volumizing skin, rebuilding skin collagen, and improving skin elasticity.

In certain instances, the method comprises applying an effective amount of a composition comprising an active agent blend topically to an area of the skin of a subject, wherein the active agent blend comprises at least one active ingredient useful for treating and/or preventing wrinkles. Active ingredients useful for treating wrinkles include but are not limited to collagen peptides, hyaluronic acid, caffeine, L-arginine HCl, vitamins A C, and E, retinoic acid, niacinamide, ceramides, beta carotene, green tea extract, licorice, oats, and soy.

### Breast Enhancement

In some embodiments, the composition is used for breast enhancement. In certain embodiments, breast enhancement refers to breast enlargement, breast firmness, and/or elasticity of the skin.

In certain instances, the method comprises applying an effective amount of a composition comprising an active agent blend topically to an area of the skin of a subject, wherein the active agent blend comprises at least one active ingredient useful for breast enhancement. Active agents useful for breast enhancement include but are not limited to botanical and herbal extracts. For example, Pueraria mirifica (aka: Kwao Krua), Fenugreek, Kigelia Africana, Dong Quai root, Motherwort, Saw Palmetto, Blessed Thistle root, Fennel seed, Kelp Powder, and Wild Yam Extract have been identified as having breast-enhancing properties. Additionally, skin enhancing and firming agents may be useful for breast enhancement, including hyaluronic acid, collagen peptides, L-arginine, L-tyrosine, and caffeine.

### Subcutaneous Fat Reduction

In some embodiments, the composition is used for subcutaneous fat reduction.

In certain instances, the method comprises applying an effective amount of a composition comprising an active agent blend topically to an area of the skin of a subject, wherein the active agent blend comprises at least one active ingredient useful for subcutaneous fat reduction. Active agents for subcutaneous fat reduction include lipolysis-enhancing agents, including yohimbine HCl, forskolin, bupleurum root, coenzyme A, and L-carnitine. Thermogenic-enhancing agents are useful in increasing lipolysis and subcutaneous fat reduction. Such agents include, but are not limited to caffeine, Capsaicin, Green tea extract, White tea extract, Oolong tea extract, Black Pepper extract (Bioperine), Green Coffee Bean extract, and Rauwolfia Vomitoria.

### Erectile Dysfunction

In some embodiments, the composition is used for treating erectile dysfunction. In certain embodiments, the method is useful for localized neurostimulation and vasodilation.

In certain instances, the method comprises applying an effective amount of a composition comprising an active agent blend topically to an area of the skin of a subject, wherein the active agent blend comprises at least one active ingredient useful for treating erectile dysfunction. Active ingredients suitable for treating erectile dysfunction include but are not limited to yohimbine HCl, caffeine, and L-arginine HCl.

### Methods of Preparing Compositions

Also disclosed are methods of preparing compositions formulated to topical application. The methods may be embodied in a variety of ways.

### Methods of Preparing a Composition for Topical Application

In one embodiment, disclosed is a method of preparing a composition formulated for topical application. In certain embodiments, the method comprises a method of preparing a composition formulated for topical application comprising: (i) preparing a carrier comprising a dual surfactant blend, (ii) preparing an active agent blend: and (iii) combining the carrier and the active agent blend. In some embodiments, the dual surfactant blend comprises benzalkonium chloride and dihydroxyethyl cocamine oxide

In some embodiments, the method comprises preparing a carrier composition comprising benzalkonium chloride and dihydroxyethyl cocamine oxide. In some embodiments the method comprises preparing one or more active agent blends. Thus, in certain embodiments, disclosed is a method of preparing any of the disclosed compositions comprising: (i) preparing a carrier comprising benzalkonium chloride and dihydroxyethyl cocamine oxide: (ii) preparing an active agent blend: and (iii) combining the carrier with the active agent blend.

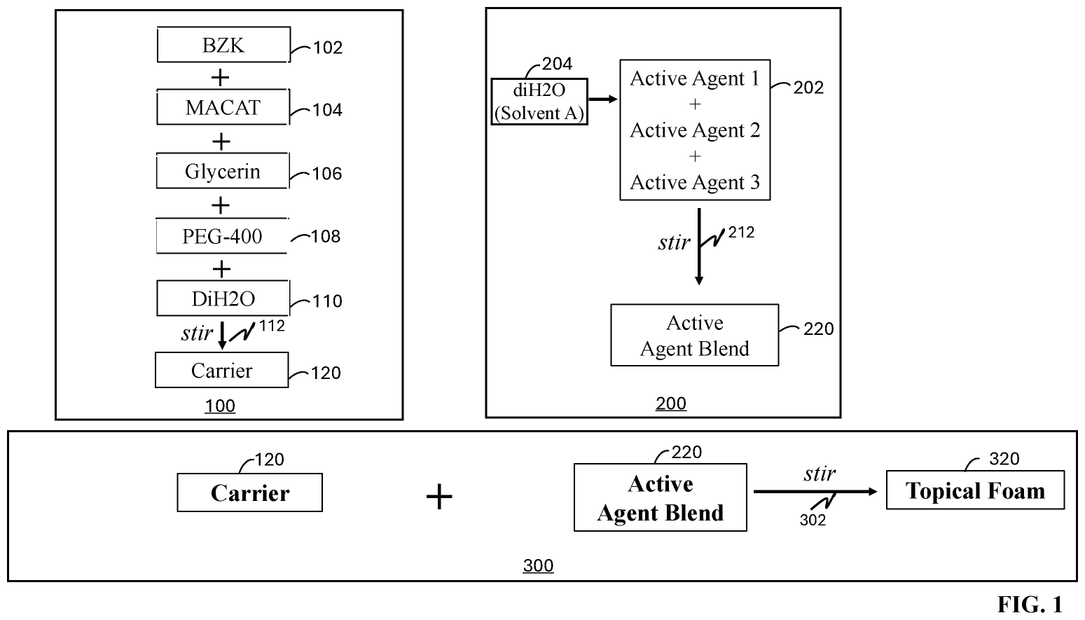

In some embodiments, the method comprises: (i) preparing a carrier comprising benzalkonium chloride and dihydroxyethyl cocamine oxide: (ii) preparing an active agent blend and (iii) combining the carrier and the active agent blend. FIG. 1 shows a non-limiting embodiment of the method of preparing a topical foam formulation. As depicted in FIG. 1, a carrier (120) may be prepared (100) by combining BZK (102), MACAT (104), glycerin (106), PEG-400 (108), and diH₂O (deionized H₂O) (110) to form a carrier composition (120).

FIG. 1 further illustrates a method (200) of preparing an active agent blend for use in the disclosed compositions. As depicted in FIG. 1, an active agent blend (220) may be prepared (200) by dissolving (212) one or more active agents (202) in a solvent (e.g., diH2O) (204).

In some embodiments, the carrier foam (120) can then be combined with the one or more active agent blends (220) to form a topical foam composition (320). In certain embodiments, the carrier (120) may be first combined with a first Active Agent Blend (220) and then combined with a second Active Agent Blend or vice versa. In certain embodiments, the two or more Active Agent Blends are combined first with each other prior to being combined with the carrier (120).

In some embodiments, combining the carrier and active agent blend requires heating. In some embodiments, the active agent blend and carrier are mixed together while being heated to at least 40, 45, 50, or 55° C. In certain instances, the carrier and active agent blend are mixed together for at least 1, 2, 3, 4, 5, 6, 7, 8, 9, 10, 11, 12, 13, 14, 15, 16, 17, 18, 19 20, 21, 22, 23, 24, or more hours.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

The following examples describe methods for the preparations and use of a topical composition.

### Example 1. Preparation of a Topical Composition

### Materials

The following materials were used in the examples and methods below:
1. Benzalkonium Chloride, CAS #68391-01-5, “Nexsurf BZK”, FW 368.0390
2. Dihydroxyethyl Cocamine Oxide, CAS #61791-47-7, “MACAT AO-12-2” Mason Chemical Co., FW 193.1977
3. Glycerol, CAS #56-81-5, FW 92.09, GRAS
4. PEG-400 (Polyethylene glycol (400) monolaurate), CAS #25322-68-3, FW 380-420,
5. Yohimbine HCl, CAS #65-19-0, FW 390.91
6. Yohimbine Bark Extract, herbal extract in Glycerin/water, Food Grade
7. Caffeine, CAS #58-08-2, FW 194.191
8. L-Arginine HCl, CAS #1119-34-2, FW 210.662
9. Collagen Peptide, Hydrolyzed Type I and III collagen, food grade (GrovyBee.com)
10. Hyaluronic Acid, CAS #9004-61-9, Food Grade-USP
11. Aloe Vera Powder, Food Grade-USP
12. Cissus quadrangularis, Food Grade
13. Forskolin, Food Grade
14. Pueraria mirifica, Food Grade
15. Fenugreek, Food Grade
16. Lidocaine HCl, CAS #6108-05-0, FW 288.81, USP

A carrier was prepared by combining benzalkonium chloride (BZK) (0.26%), dihydroxyethyl cocamine oxide (MACAT) (0.26%), glycerol (0.5%) PEG-400 (2.0%), and deionized (DiH2O) (89.96%) into a bottle. The solution was then mixed.

Next, the active agent blend solution was prepared. In a first formulation (TABLE 1), L-Arginine (6.0%), caffeine (1.0%), and yohimbine HCl (0.2%) were combined and the solution was shaken to dissolve the powder.

Using the method described above, various formulations were prepared according to the specifications provided in TABLES 2-9. Heating is not required but can speed up the mixing process. The solution should not be heated to higher than 60° C. during production. Filtration (macro) may be needed for some compositions that contain herbal ingredients that contain insoluble plant materials.
Formulation #1 (TABLE 1) (ED045) is useful for health and fitness.
Formulation #2 (TABLE 2) (ED025) is useful for health and fitness and treatment of erectile dysfunction.
Formulation #3 (TABLE 3) (ED046) is useful for pain relief.
Formulation #4 (TABLE 4) (ED042) is useful for pain relief.
Formulation #5 (TABLE 5) (JF003) is useful for joint health and repair.
Formulation #6 (TABLE 6) (ED033) is useful for wrinkle treatment.
Formulation #7 (TABLE 7) (ED040) is useful for breast enhancement.
Formulation #8 (TABLE 8) (LF009) is useful for subcutaneous fat reduction.
Formulation #9 (TABLE 9) (LF007) is useful for subcutaneous fat reduction.
Formulation #10 (TABLE 10) LID is useful for pain relief and topical anesthesia.

TABLE 1

| | | | % total |
|---|---|---|---|
| Formulation | NBC-ED-045 | | weight of |
| # 1 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 899.6 | 89.96 |
| Active Agent | L-Arginine | 60 | 6.0 |
| Blend | Caffeine | 10 | 1.0 |
| | Yohimbine Bark | 0.2 | 0.02 |
| | Extract | | |

TABLE 2

| | | | % total |
|---|---|---|---|
| Formulation | NBC-ED-025 | | weight of |
| # 2 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 897.8 | 89.78 |
| Active Agent | L-Arginine | 60 | 6.0 |
| Blend | Caffeine | 10 | 1.0 |
| | Yohimbine HCl | 2 | 0.2 |

TABLE 3

| | | | % total |
|---|---|---|---|
| Formulation | NBC-ED-046 | | weight of |
| # 3 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 896.6 | 89.66 |
| Active Agent | L-Arginine | 60 | 6.0 |
| Blend | Caffeine | 10 | 1.0 |
| | Cissus quadrangularis | 3 | 0.3 |
| | Yohimbine Bark | 0.2 | 0.02 |
| | Extract | | |

TABLE 4

| | | | % total |
|---|---|---|---|
| Formulation | NBC-ED-042 | | weight of |
| # 4 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 892.8 | 89.28 |
| Active Agent | L-Arginine | 60 | 6.0 |
| Blend | Caffeine | 10 | 1.0 |
| | Yohimbine HCl | 2 | 0.2 |

TABLE 5

| | | | % total |
|---|---|---|---|
| Formulation | NBC- JF003 | | weight of |
| # 5 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 889.8 | 88.98 |
| Active Agent | L-Arginine | 60 | 6.0 |
| Blend | Caffeine | 10 | 1.0 |
| | Collagen Peptides | 5 | 0.5 |
| | Cissus Q | 3 | 0.3 |
| | Yohimbine HCl | 2 | 0.2 |

TABLE 6

| | | | % total |
|---|---|---|---|
| Formulation | NBC-ED-033 | | weight of |
| # 6 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 892.8 | 89.28 |
| Active Agent | L-Arginine | 60 | 6.0 |
| Blend | Caffeine | 10 | 1.0 |
| | Collagen Peptides | 5 | 0.5 |
| | Hyaluronic Acid | 1 | 0.1 |
| | Aloe Vera Powder | 1 | 0.1 |

TABLE 7

| | | | % total |
|---|---|---|---|
| Formulation | NBC-ED-040 | | weight of |
| # 7 | Materials: | g/L | composition |
| Carrier: | BZK | 2.6 | 0.26 |
| | MACAT | 2.6 | 0.26 |
| | Glycerin | 5.0 | 0.5 |
| | PEG-400 | 20 | 2.0 |
| | DiH2O | 897.8 | 89.78 |
| Active Agent | L-Arginine | 60 | 6.0 |
| Blend | Caffeine | 10 | 1.0 |
| | Collagen Peptides | 5 | 0.5 |
| | Hylauronic Acid | 0.1 | 0.01 |
| | Pueraria mirifica | 6 | 0.6 |
| | Fenugreek | 3 | 0.3 |

TABLE 8

| | | | % total |
|---|---|---|---|
| Formulation | NBC- LF009 | | weight of |
| #8 | Materials: | g/L | composition |
| Carrier: | BZK | 2.6 | 0.26 |
| | MACAT | 2.6 | 0.26 |
| | Glycerin | 5 | 0.5 |
| | PEG-400 | 20 | 2.0 |
| | DiH2O | 831.7 | 83.17 |
| Active Agent | L-Arginine | 60 | 6.0 |
| Blend | Caffeine | 20 | 2.0 |
| | Cissus quadrangularis | 3 | 0.3 |
| | Yohimbine Bark | 0.1 | 0.01 |
| | Extract | | |
| | Forskolin | 5 | 0.5 |
| | COAXEL ™ PH | 50 | 5.0 |

TABLE 9

| | | | % total |
|---|---|---|---|
| Formulation | NBC- LF007 | | weight of |
| # 9 | Materials: | g/L | composition |
| Carrier: | BZK | 2.6 | 0.26 |
| | MACAT | 2.6 | 0.26 |
| | Glycerin | 5 | 0.5 |
| | PEG-400 | 20 | 2.0 |
| | DiH2O | 874.8 | 87.48 |
| Active Agent | Caffeine | 20 | 2.0 |
| Blend | Forskolin | 5 | 0.5 |
| | Cissus Q | 10 | 1.0 |
| | COAXEL ™ PH | 50 | 5.0 |
| | Yohimbine HCl | 10 | 1.0 |

TABLE 10

| | | | % total |
|---|---|---|---|
| Formulation | NBC- Lido001 | | weight of |
| # 10 | Materials: | g/L | composition |
| Carrier: | BZK | 2.6 | 0.26 |
| | MACAT | 2.6 | 0.26 |
| | Glycerin | 5 | 0.5 |
| | PEG-400 | 20 | 2.0 |
| | DiH2O | 869.8 | 86.98 |
| Active Agent | Lidocaine HCl | 100 | 10.0 |
| Blend | | | |

### Example 2. Formulation Testing

Each, formulation was tested to determine whether its combination of active ingredients was (1) soluble, (2) capable of foaming, and (3) stable under various conditions. The results of these tests are presented in TABLES 11-14.

The solubility of each ingredient was assessed. For each active agent, a percent loading at which each ingredient stops totally dissolving in the carrier composition was determined. The addition of other active agents can affect the maximum level of solubility for each active ingredient and disrupt the ability of the formulation to form a foam. Thus, each formulation was tested for the ability of its ingredients to dissolve in the carrier composition at various temperatures: cool (˜4° C.) (TABLE 11), room temperature (˜20-24° C.) (TABLE 12), and warm (˜55° C.) (TABLE 13). Each of the active agent blends were able to dissolve into the carrier composition across temperatures of 4-55° C. as determined by a lack of precipitation.

The formulations were further assessed for foaming capability. The base carrier composition was designed to produce a foam that would equal that of non-alcohol based hand sanitizer products in consistency, density, and feel. The foam formulations should not be loose and runny upon pumping and should be able to be pumped and inverted and still be able to stay firm and suspended instead of drip and fall off a surface (such as an individual's hand). Each formulation was tested for its ability to form a foam (i.e., stay firm and be inverted without running/dripping). Each of the formulations were able to form a foam and remain as a foam across temperatures of 4-55° C. (TABLES 11-13).

Additionally, formulation stability was assessed under various conditions. Stable foam formulations stay blended without clouding due to precipitation of ingredients, crystal formation over time, color changes, and/or separation layers. Thus, formulations were observed during testing for any changes in appearance and/or consistency. All stability tests were performed in a closed container and preferably done in an end-use bottle/container to mimic an end-user experience. Formulation stability was assessed in both light and dark environments to evaluate any light (normally UV) sensitivity (data not shown). Any light sensitivity can be and was overcome using packaging materials generally known in the art for light protection. Testing of formulation stability was also assessed at various temperatures: cool (˜4° C.) (TABLE 11), room temperature (˜20-24° C.) (TABLE 12), and warm (˜55° C.) (TABLE 13). All formulations remained stable across temperatures of 4-55° C. as determined by the lack of clouding due to precipitation of ingredients, crystal formation over time, color changes, and separation layers.

Additionally, formulation stability was assessed across one or more freeze-thaw cycles to evaluate any solubility issues following a freeze-thaw cycle (TABLE 14). Following a freeze-thaw cycle, formulations were observed for any solubility issues. All of the formulations were stable (i.e., remained clear and transparent without clouding due to precipitation of ingredients, crystal formation over time, color changes, and/or separation layers) (TABLE 14). Additionally, all formulations were able to blend back together after thawing with minimal effort (e.g., gentle mixing) (TABLE 14).

TABLE 11
Formulation Solubility, Foaming Ability, and Stability at 4° C.

| | | Foaming | |
|---|---|---|---|
| | Solubility | Ability | Stability |
| Formulation | (Yes/No) | (Yes/No) | (Yes/No) |
| NBC-ED045 | Yes | Yes | Yes |
| (TABLE 1) | | | |
| NBC-ED025 | Yes | Yes | Yes |
| (TABLE 2) | | | |
| NBC-ED046 | Yes | Yes | Yes |
| (TABLE 3) | | | |
| NBC-JED042 | Yes | Yes | Yes |
| (TABLE 4) | | | |
| NBC-JF003 | Yes | Yes | Yes |
| (TABLE 5) | | | |
| NBC-ED033 | Yes | Yes | Yes |
| (TABLE 6) | | | |
| NBC-ED040 | Yes | Yes | Yes |
| (TABLE 7) | | | |
| NBC-LF009 | Yes | Yes | Yes |
| (TABLE 8) | | | |
| NBC-LF007 | Yes | Yes | Yes |
| (TABLE 9) | | | |
| NBC-Lido001 | Yes | Yes | Yes |
| (TABLE 10) | | | |

TABLE 12
Formulation Stability at 20-24° C.

| | | Foaming | |
|---|---|---|---|
| | Solubility | Ability | Stability |
| Formulation | (Yes/No) | (Yes/No) | (Yes/No) |
| NBC-ED045 | Yes | Yes | Yes |
| (TABLE 1) | | | |
| NBC-ED025 | Yes | Yes | Yes |
| (TABLE 2) | | | |
| NBC-ED046 | Yes | Yes | Yes |
| (TABLE 3) | | | |
| NBC-JED042 | Yes | Yes | Yes |
| (TABLE 4) | | | |
| NBC-JF003 | Yes | Yes | Yes |
| (TABLE 5) | | | |
| NBC-ED033 | Yes | Yes | Yes |
| (TABLE 6) | | | |
| NBC-ED040 | Yes | Yes | Yes |
| (TABLE 7) | | | |
| NBC-LF009 | Yes | Yes | Yes |
| (TABLE 8) | | | |
| NBC-LF007 | Yes | Yes | Yes |
| (TABLE 9) | | | |
| NBC-Lido001 | Yes | Yes | Yes |
| (TABLE 10) | | | |

TABLE 13
Formulation Stability at 55° C.

| | | Foaming | |
|---|---|---|---|
| | Solubility | Ability | Stability |
| Formulation | (Yes/No) | (Yes/No) | (Yes/No) |
| NBC-ED045 | Yes | Yes | Yes |
| (TABLE 1) | | | |
| NBC-ED025 | Yes | Yes | Yes |
| (TABLE 2) | | | |
| NBC-ED046 | Yes | Yes | Yes |
| (TABLE 3) | | | |
| NBC-JED042 | Yes | Yes | Yes |
| (TABLE 4) | | | |
| NBC-JF003 | Yes | Yes | Yes |
| (TABLE 5) | | | |
| NBC-ED033 | Yes | Yes | Yes |
| (TABLE 6) | | | |
| NBC-ED040 | Yes | Yes | Yes |
| (TABLE 7) | | | |
| NBC-LF009 | Yes | Yes | Yes |
| (TABLE 8) | | | |
| NBC-LF007 | Yes | Yes | Yes |
| (TABLE 9) | | | |
| NBC-Lido001 | Yes | Yes | Yes |
| (TABLE 10) | | | |

TABLE 14
Freeze/Thaw: −20° C. to Room Temperature (~22° C.)

| | Freeze/thaw stability |
|---|---|
| Formulation | (−20° C.-22° C.) |
| NBC-ED045 | Pass |
| (TABLE 1) | |
| NBC-ED025 | Pass |
| (TABLE 2) | |
| NBC-ED046 | Pass |
| (TABLE 3) | |
| NBC-JED042 | Pass |
| (TABLE 4) | |
| NBC-JF003 | Pass |
| (TABLE 5) | |
| NBC-ED033 | Pass |
| (TABLE 6) | |
| NBC-ED040 | Pass |
| (TABLE 7) | |
| NBC-LF009 | Pass |
| (TABLE 8) | |
| NBC-LF007 | Pass |
| (TABLE 9) | |
| NBC-Lido001 | Pass |
| (TABLE 10) | |

### Example 3. Application and Delivery Efficiency

Application and delivery efficiency were tested by evaluating skin applications. Each formulation was evaluated post-application to the skin of a subject by examining the application area for a period of time following application.

Unsuccessful delivery results in residue being deposited on the skin instead of being transdermally delivered into the skin tissue. The presence of any undelivered material indicates that the formulation was not able to transdermally deliver one or more active agents. An unsuccessful product application will result in any of the active ingredients not being absorbed into the skin, which will result on a white paste, flakes, or powdery deposit on the skin surface once the application has dried. A successful product application may initially feel tacky but will dry and be visually clear within a few (i.e., 5 mins). Some active agents and/or functional agents may alter the speed at which the application site dries or may show a persistent tackiness over time. All formulations disclosed in TABLES 1-9 were found to successfully deliver the active and functional agents without residue being deposited on the skin.

### Example 4. Topical Use of Formulations NBC-ED025 and NBC-JF003 for Pain Relief

For pain relief, beta testers experiencing pain in various areas of their body (e.g., hip, knee, lower back, head, hand, and wrist) were instructed to use Formulation #NBC-ED025 or NBC-JF003 as needed for pain. Beta testers experienced pain from a variety of causes including osteoarthritis, neuropathic pain (caused by multiple sclerosis), degenerative disc disease, lumbosacral spondylosis, acute pain from injuries, burns, injections, nociceptive pain, and migraines.

100% of beta testers indicated they achieved equal or better pain relief using the topical composition on localized body parts where they regularly experienced pain that they normally treated with oral pain relievers. Beta testers also indicated that they preferred the formulations which does not leave a greasy or sticky residue on the surface of the skin. Beta testers reported shortened time for pain relief (e.g. <1, 3, or 5 mins) as compared to 20-30 mins when using oral pain killers (˜20-30 mins). Beta testers for pain relief reported that pain relief lasted from 6-10 hours following topical application.

### Beta Tester #1: Pain Relief

Beta test #1 was a subject who has experienced osteoarthritis in her right hip for approximately 40 years. Beta tester #1 also has experienced pain in her wrist that is unresponsive to pain medications. Beta Tester #1 was given formulation NBC-JF003 (TABLE 4) and was instructed to apply as needed to treat pain. Beta Tester #1 applied formulation NBC-JF003 (TABLE 4) to the area of her skin covering her hip and/or wrist as she experienced pain over a 6 month period. Beta Tester #1 reported that pain relief for treatment of wrist pain that did not respond to other medications occurred within 3-5 minutes of application, and lasted for hours, and that the formulation is one she would use in the future as a primary tool for pain management.

### Beta Tester #2: Pain Relief

Beta Tester #2 was a subject who experiences chronic pain from multiple sclerosis, persistent back pain, osteoarthritis in both knees, and migraines. Beta tester #2 also experiences nociceptive pain in his right hand due to a crushing injury. His nociceptive pain was described as a throbbing, achy pain with skin tingling and numbness sensation. He further described that his right-hand pain is often felt in the skin, tendons, and muscles. Beta Tester #2 also described experiencing occasional acute pain due to injuries, burns, and injection sites. Beta Tester #2 applied formulation NBC-JF003 (TABLE 4) or NBC-ED025 (TABLE 2) to the area of his skin where he was experiencing pain. Beta Tester #2 reported pain relief was similar for both NBC-JF003 (TABLE 4) and NBC-ED025 (TABLE 2). Beta Tester #2 reported that pain relief lasted approximately 2 hours longer following treatment with NBC-ED025 (TABLE 2) as compared to NBC-JF003 (TABLE 4).

Additionally, Beta Tester #2 reported the following after using formulations NBC-JF003 (TABLE 4) and NBC-ED025 (TABLE 2). For physiological symptoms that produce a multitude of everyday challenges and pain types, SYNAFOAM™ has been extremely effective, allowing for a reduction and/or complete cessation of the pain, almost eliminating the use of other pain medication (including over the counter analgesics, epidural shots and opiates) thereby reducing the side-effects of other medications. Additionally, Beta Tester #2 noted that as compared to 20-30 min for oral painkillers, SYNAFOAM™ acts within 60 seconds or less with total relief occurring in under five minutes. Beta Tester #2 also provided that pain relief lasted for about six to ten hours following treatment with SYNAFOAM™.

### Example 5. Topical Use of Formulation NBC-ED025 for Enhanced Athletic Performance

This disclosure includes, but is not limited to, the following embodiments.

For enhanced athletic performance, beta testers were instructed to use formulation NBC-ED025 (TABLE 2) as desired. Beta testers liberally applied the formulation to sections of the skin (e.g., arms, legs, back, abdominals) prior to an athletic activity (e.g., a workout). Beta testers for athletic performance reported the following positive effects: decreased warm-up times required prior to an athletic event and decreased recovery time following an athletic event.

### Beta Tester #3: Enhanced Athletic Performance

Beta Tester #3 is a fitness expert and personal trainer seeking to enhance athletic performance. Beta Tester #3 was given formulation NBC-ED025 (TABLE 2) and was instructed to apply as desired prior to and/or after athletic activity. Beta Tester #3 noted that the foam worked extremely well, and when applied prior to a workout, resulted in feeling already warmed up and ready to go, with a decrease in recovery time.

### Example 6. Embodiments of Suitable Compositions and Methods

This disclosure includes, but is not limited to, the following embodiments.
A.1 A composition formulated for topical application comprising a carrier composition comprising a dual surfactant blend and a therapeutically effective amount of an active agent blend.
A.2. The composition of embodiment A.1, wherein the dual surfactant blend comprises benzalkonium chloride and dihydroxyethyl cocamine oxide.
A.3 The composition of embodiment A. 1-A.2, wherein the carrier further comprises from about 0.2% to 0.3% by weight of benzalkonium chloride.
A.4 The composition of embodiments A.1-A.3, wherein the composition comprises from about 0.2% to 0.3% by weight of dihydroxyethyl cocamine oxide.
A.5 The composition of embodiments A.1-A.4, wherein the active agent blend comprises at least one active ingredient for improving health and fitness.
A.6 The composition of embodiments A.1-A.5, wherein the active agent blend comprises at least one active ingredient for preventing and/or relieving pain.
A.7 The composition of embodiments A.1-A.6, wherein the active agent blend comprises at least one active ingredient for joint and health repair.
A.8 The composition of embodiments A.1-A.7, wherein the active agent blend comprises at least one active ingredient for preventing and/or treating wrinkles.
A.9 The composition of embodiments A.1-A.8, wherein the active agent blend comprises at least one active ingredient for breast enhancement.
A.10 The composition of embodiments A. 1-A.9, wherein the active agent blend comprises at least one active ingredient for subcutaneous fat reduction.
A.11 The composition of embodiments A. 1-A.10, wherein the active agent blend comprises at least one active ingredient for treating and/or preventing erectile dysfunction.
A.12 The composition of embodiments A.1-A.11, wherein the active agent blend comprises L-Arginine, caffeine, and yohimbine HCl.
A.13 The composition of embodiments A.1-A. 12, wherein the active agent blend comprises L-Arginine and caffeine.
A.14 The composition of embodiments A.1-A.13, wherein the active agent blend comprises L-Arginine, caffeine, collagen peptides, Cissus quadrangularis, and yohimbine.
A.15 The composition of embodiments A.1-A.14, wherein the active agent blend comprises L-Arginine, caffeine, collagen peptides, hyaluronic acid, and aloe vera powder.
A.16 The composition of embodiments A.1-A.15, wherein the active agent blend comprises L-Arginine, caffeine, collagen peptides, hyaluronic acid, Pueraria mirifica, and fenugreek.
A.17 The composition of embodiments A.1-A.16, wherein the active agent blend comprises L-Arginine, caffeine, Cissus quadrangularis, yohimbine, forskolin, and Coaxel.
A.18 The composition of embodiments A.1-A.17, wherein the active agent blend comprises caffeine, yohimbine, forskolin, Cissus quadrangularis, and Coaxel.
A.19 The composition of embodiments A.1-A.18, wherein the active agent blend comprises lidocaine HCl.
A.20 The composition of embodiments A.1-A.19, wherein the composition comprises less than about 10% by weight of the active agent blend.
A.21 The composition of embodiments A.1-A.20, wherein the carrier further comprises water or buffered water.
A.22 The composition of embodiments A.1-A.21, wherein the carrier further comprises glycerin.
A.23 The composition of embodiments A.1-A.22, wherein the carrier further comprises PEG-400.
A.24 The composition of embodiments A.1-A.23, wherein the composition is a foam.
B.1 A method of using any of the compositions of embodiments of A.1-A.24 comprising topically administering the composition to the skin of a subject thereby delivering at least one active agent across the skin.
B.2 The method of embodiment B.2, wherein the composition is formulated for topical application comprising a carrier composition comprising a dual surfactant blend and a therapeutically effective amount of an active agent blend.
B.3 The method of embodiments B.1-B.2, wherein the dual surfactant blend comprises benzalkonium chloride and dihydroxyethyl cocamine oxide.
B.4 The method of embodiments B.1-B.3, the carrier further comprises from about 0.2% to 0.3% by weight of benzalkonium chloride.
B.5 The method of embodiments B.1-B.4, wherein the composition comprises from about 0.2% to 0.3% by weight of dihydroxyethyl cocamine oxide.
B.6 The method of embodiments B.1-B.5, wherein the active agent blend comprises at least one active ingredient for improving health and fitness.
B.7 The method of embodiments B.1-B.6, wherein the active agent blend comprises at least one active ingredient for preventing and/or relieving pain.
B.8 The method of embodiments B.1-B.7, wherein the active agent blend comprises at least one active ingredient for joint and health repair.
B.9 The method of embodiments B.1-B.8, wherein the active agent blend comprises at least one active ingredient for preventing and/or treating wrinkles.
B.10 The method of embodiments B.1-B.9, wherein the active agent blend comprises at least one active ingredient for breast enhancement.
B.11 The method of embodiments B.1-B. 10, wherein the active agent blend comprises at least one active ingredient for subcutaneous fat reduction.
B.12 The method of embodiments B.1-B.11, wherein the active agent blend comprises at least one active ingredient for treating and/or preventing erectile dysfunction.
B.13 The method of embodiments B.1-B. 12, wherein the active agent blend comprises L-Arginine, caffeine, and yohimbine HCl.
B.14 The method of embodiments B.1-B.13, wherein the active agent blend comprises L-Arginine and caffeine.
B.15 The method of embodiments B.1-B.14, wherein the active agent blend comprises L-Arginine, caffeine, collagen peptides, Cissus quadrangularis, and yohimbine.
B.16 The method of embodiments B.1-B.15, wherein the active agent blend comprises L-Arginine, caffeine, collagen peptides, hyaluronic acid, and aloe vera powder.
B.17 The method of embodiments B.1-B.16, wherein the active agent blend comprises L-Arginine, caffeine, collagen peptides, hyaluronic acid, Pueraria mirifica, and fenugreek.
B.18 The method of embodiments B.1-B.17, wherein the active agent blend comprises L-Arginine, caffeine, Cissus quadrangularis, yohimbine, forskolin, and Coaxel.
B.19 The method of embodiments B.1-B.18, wherein the active agent blend comprises caffeine, yohimbine, forskolin, Cissus quadrangularis, and Coaxel.
B.20 The method of embodiments B.1-B. 19, wherein the active agent blend comprises lidocaine HCl.
B.21 The method of embodiments B.1-B.20, wherein the composition comprises less than about 10% by weight of the active agent blend.
B.22 The method of embodiments B.1-B.21, wherein the carrier further comprises water or buffered water.
B.23 The method of embodiments B.1-B.22, wherein the carrier further comprises glycerin.
B.24 The method of embodiments B.1-B.23, wherein the carrier further comprises PEG-400.
B.25 The method of embodiments B.1-B.24, wherein the composition is a foam.
C.1 A method of preparing any of the compositions of claims A.1-A.24 comprising: (i) preparing a carrier composition comprising a dual surfactant blend: (ii) preparing an active agent blend: and (iii) combining the carrier with the active agent blend.
C.2 The method of embodiment C.1, wherein the composition is formulated for topical application comprising a carrier composition comprising a dual surfactant blend and a therapeutically effective amount of an active agent blend.
C.3 The method of embodiments C.1-C.2, wherein the dual surfactant blend comprises benzalkonium chloride and dihydroxyethyl cocamine oxide.
C.4 The method of embodiments C.1-C.3, the carrier comprises from about 0.2% to 0.3% by weight of benzalkonium chloride.
C.5 The method of embodiments C.1-C.4, wherein the composition comprises from about 0.2% to 0.3% by weight of dihydroxyethyl cocamine oxide.
C.6 The method of embodiments C.1-C.5, wherein the active agent blend comprises at least one active ingredient for improving health and fitness.
C.7 The method of embodiments C.1-C.6, wherein the active agent blend comprises at least one active ingredient for preventing and/or relieving pain.
C.8 The method of embodiments C.1-C.7, wherein the active agent blend comprises at least one active ingredient for joint and health repair.
C.9 The method of embodiments C.1-C.8, wherein the active agent blend comprises at least one active ingredient for preventing and/or treating wrinkles.
C.10 The method of embodiments C.1-C.9, wherein the active agent blend comprises at least one active ingredient for breast enhancement.
C.11 The method of embodiments C.1-C.10, wherein the active agent blend comprises at least one active ingredient for subcutaneous fat reduction.
C.12 The method of embodiments C.1-C.11, wherein the active agent blend comprises at least one active ingredient for treating and/or preventing erectile dysfunction.
C.13 The method of embodiments C.1-C.12, wherein the active agent blend comprises L-Arginine, caffeine, and yohimbine HCl.
C.14 The method of embodiments C.1-C.13, wherein the active agent blend comprises L-Arginine and caffeine.
C.15 The method of embodiments C.1-C.14, wherein the active agent blend comprises L-Arginine, caffeine, collagen peptides, Cissus quadrangularis, and yohimbine.
C.16 The method of embodiments C.1-C.15, wherein the active agent blend comprises L-Arginine, caffeine, collagen peptides, hyaluronic acid, and aloe vera powder.
C.17 The method of embodiments C.1-C.16, wherein the active agent blend comprises L-Arginine, caffeine, collagen peptides, hyaluronic acid, Pueraria mirifica, and fenugreek.
C.18 The method of embodiments C.1-C.17, wherein the active agent blend comprises L-Arginine, caffeine, Cissus quadrangularis, yohimbine, forskolin, and Coaxel.
C.19 The method of embodiments C.1-C.18, wherein the active agent blend comprises caffeine, yohimbine, forskolin, Cissus quadrangularis, and Coaxel.
C.20 The method of embodiments C.1-C.19, wherein the active agent blend comprises lidocaine HCl.
C.21 The method of embodiments C.1-C.20, wherein the composition comprises less than about 10% by weight of the active agent blend.
C.22 The method of embodiments C.1-C.21, wherein the carrier further comprises water or buffered water.
C.23 The method of embodiments C.1-C.22, wherein the carrier further comprises glycerin.
C.24 The method of embodiments C.1-C.23, wherein the carrier further comprises PEG-400.
C.25 The method of embodiments C.1-B.24, wherein the composition is a foam.
C26. The method of embodiments C.1-B.24, wherein the dual surfactant blend comprises benzalkonium chloride and dihydroxyethyl cocamine oxide.

What is claimed:

1. A composition formulated for topical application comprising:
(i) a carrier composition comprising a dual surfactant blend; and
(ii) a therapeutically effective amount of an active agent blend.

2. The composition of claim 1, wherein the dual surfactant blend comprises benzalkonium chloride and dihydroxyethyl cocamine oxide.

3. The composition of claim 2, wherein the carrier comprises from about 0.2% to 0.3% by weight of benzalkonium chloride.

4. The composition of claim 2, wherein the carrier comprises from about 0.2% to 0.3% by weight of dihydroxyethyl cocamine oxide.

5. The composition of claim 1, wherein the active agent blend comprises at least one active ingredient for improving health and fitness.

6. The composition of claim 1, wherein the active agent blend comprises at least one active ingredient for preventing and/or relieving pain.

7. The composition of claim 1, wherein the active agent blend comprises at least one active ingredient for joint and health repair.

8. The composition of any claim 1, wherein the active agent blend comprises at least one active ingredient for preventing and/or treating wrinkles.

9. The composition of claim 1, wherein the active agent blend comprises at least one active ingredient for breast enhancement.

10. The composition of claim 1, wherein the active agent blend comprises at least one active ingredient for subcutaneous fat reduction.

11. The composition of claim 1, wherein the active agent blend comprises at least one active ingredient for treating and/or preventing erectile dysfunction.

12. The composition of claim 1, wherein the active agent blend comprises L-Arginine, caffeine, and yohimbine.

13. The composition of any one of claim 1, wherein the active agent blend comprises L-Arginine, caffeine, Cissus quadrangularis, and yohimbine.

14. The composition of claim 1, wherein the active agent blend comprises L-Arginine, caffeine, collagen peptides, Cissus quadrangularis, and yohimbine.

15. The composition of claim 1, wherein the active agent blend comprises L-Arginine, caffeine, collagen peptides, hyaluronic acid, and aloe vera powder.

16. The composition of claim 1, wherein the active agent blend comprises L-Arginine, caffeine, collagen peptides, hyaluronic acid, Pueraria mirifica, and fenugreek.

17. The composition of claim 1, wherein the active agent blend comprises L-Arginine, caffeine, Cissus quadrangularis, yohimbine, forskolin, and Coaxel.

18. The composition of claim 1, wherein the active agent blend comprises, caffeine, yohimbine, forskolin, Cissus quadrangularis, and Coaxel.

19. The composition of claim 1, wherein the active agent blend comprises, lidocaine HCl.

20. The composition of claim 1, wherein the composition comprises less than about 10% by weight of the active agent blend.

21. The composition of claim 1, wherein the carrier further comprises water or buffered water.

22. The composition of any one of claim 1, wherein the carrier further comprises glycerin.

23. The composition of claim 1, wherein the carrier further comprises PEG-400.

24. The composition of claim 1, wherein the composition is a foam.

25. A method of using a composition formulated for topical application comprising topically administering the composition to the skin of a subject thereby delivering at least one active agent across the skin, wherein the composition comprises:
(i) a carrier composition comprising a dual surfactant blend, and
(ii) an active agent blend.

26. The method of claim 25, wherein the dual surfactant blend comprises benzalkonium chloride and dihydroxyethyl cocamine oxide.

27. The method of claim 25, wherein the active agent blend comprises one or more of L-Arginine, caffeine, Yohimbine HCl, Yohimbine bark extract, collagen peptides, hyaluronic acid, aloe vera powder, Cissus quadrangularis, Pueraria mirifica, fenugreek, forskolin, Coaxel pH, or lidocaine HCl.

28. A method of preparing a composition formulated for topical application comprising:
(i) preparing a carrier composition comprising a dual surfactant blend;
(ii) preparing an active agent blend; and
(iii) combining the carrier with the active agent blend.

29. The method of claim 28, wherein the dual surfactant blend comprises benzalkonium chloride and dihydroxyethyl cocamine oxide.

30. The method of claim 28, wherein the active agent blend comprises one or more of L-Arginine, caffeine, Yohimbine HCl, Yohimbine bark extract, collagen peptides, hyaluronic acid, aloe vera powder, Cissus quadrangularis, Pueraria mirifica, fenugreek, forskolin, Coaxel pH, or lidocaine HCl.
